(19) United States
(12) Patent Application Publication
(10) Pub. No.: US 2004/0076694 A1
(43) Pub. Date: Apr. 22, 2004

(54) Peppermint isopropyl alcohol

(76) Inventor: Anne M. Lia, New York, NY (US)

Correspondence Address: Anne M. Lia, Apartment 605, 340 West 85th Street, New York, NY, 10024-3804, US

(21) Appl. No.: 10/271,905

(22) Filed: Oct. 16, 2002

(51) Int. Cl. A61K 35/78; A61K 31/045

(52) U.S. Cl. 424/747; 514/724

(57) ABSTRACT

An improved isopropyl alcohol compound contains the cooling, soothing, itch-relieving, antibacterial, antifungal and deodorizing properties and herbal fragrance of pure, natural peppermint oil. Its ideal uses are for foot deodorizing and therapy, sanitizing and deodorizing the insides of shoes, for oily hair and scalp and dandruff therapy, spa treatment massage, the removal of body perspiration and odor in areas that are not sensitive to isopropyl alcohol. The compound is identifiable from other alcohol compounds by its pink color, produced by small amounts of FD&C natural liquid colorings.

### CROSS REFERENCES TO RELATED APPLICATIONS

Not Applicable

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

The invention is a multi-purpose toiletry, therapeutic medicinal remedy, herbal rubefacient, and body, foot and shoe cleanser and deodorizer for children and adults.

Antimicrobial, cleansing, rubefacient, freshening and deodorizing toiletries are necessary for physical and mental well being and self care. Freshening, cleansing and sanitizing the linings of shoes is a necessary precaution for the control of foot and shoe odor and infection.

Previous and current uses of isopropyl alcohol are for the purposes of sanitizing and cleansing the surface of the skin with an antimicrobial solution. Isopropyl alcohol is added to bathing and soaking solutions to provide antimicrobial therapy and help cleanse the body.

Plain isopropyl alcohol does not contain a special ingredient that adds antibacterial, deodorizing, cooling, soothing, energizing and itch-relieving therapy to the skin, hair and scalp. It does not leave a fresh, clean herbal scent when used to clean foot and body skin, scalp and shoe linings.

Pure, natural peppermint oil has been used as a cooling, antibacterial, medicinal and therapeutic remedy for spa treatment massages, muscle relaxation, fungus infections and deodorizing, and as an herbal fragrance.

### OBJECTS AND ADVANTAGES OF THE INVENTION

Besides the usual sanitizing, antimicrobial, cleansing and rubefacient properties of plain isopropyl alcohol, the invention has the following advantages.

a. It provides the cooling, stimulating, antifungal, antibacterial and itch-relieving effects of peppermint to the skin and scalp.

b. It deodorizes the skin and scalp and removes perspiration and odor by cleansing with a natural peppermint fragrance.

c. It cleans and deodorizes the linings of shoes, boots, sneakers and slippers and leaves a pleasant peppermint fragrance.

d. It dissolves and dilutes peppermint oil to render it less concentrated, less irritating, less potent and non-oily, so it may be safely and comfortably used for its therapeutic, antifungal and deodorizing properties without leaving an oily residue.

### BRIEF SUMMARY OF THE INVENTION

Peppermint Isopropyl Alcohol is composed of ¼ teaspoon (1.25 ml or 15 drops) of pure, natural peppermint oil to one pint (16 oz. or 473 ml) of 70% to 91% isopropyl alcohol. It is colored with 3 drops of FD&C coloring per pint, extracted from a mixture of 3 drops of FD&C reds 40 and 3 and 1 drop of FD&C blue 1. Other FD&C red and blue colorings may be used to color the compound. The invention is used as a freshener, cleanser, rubefacient and deodorizer for foot and body skin, hair, scalp and the linings of shoes, boots, sneakers and slippers.

### DESCRIPTION OF THE DRAWINGS

Not Applicable

### DETAILED DESCRIPTION AND OPERATION OF INVENTION

The proper production of Peppermint Isopropyl Alcohol is achieved by mixing ¼ teaspoon (1.25 ml or 15 drops) of pure, natural peppermint oil into one pint (16 oz. or 473 ml) of 70% to 91% isopropyl alcohol. The solution is safely and naturally colored by mixing in 3 drops of solution made with 3 drops of FD&C reds 40 and 3 and FD&C blue 1. The solution in its bottle should be shaken well before each use.

The invention is a toiletry preparation that may be used as a foot freshener and deodorizer and an additive to foot soaking solutions for a soothing and stimulating foot bath or foot soak. It is recommended as an additive to the podiatrist's pre-surgical whirlpool treatment. The invention may be applied full strength to dry (not wet) feet with cotton puffs after bathing or soaking or at any time, especially to feet bottoms and between and around the toes, to relieve burning and itching. The invention is a useful and helpful alternative to oily and/or creamy foot lotions or balms for individuals who have foot perspiration and odor problems or must avoid products that hold moisture due to foot fungus. The product relieves the itching of foot fungus, removes fungus killed by medication and prepares the skin for medication.

Peppermint Isopropyl Alcohol may be used on soft facial tissue to remove body perspiration and odor and deodorize the skin to prepare it for the application of deodorant. The invention is not intended to be used in place of or as a substitute for deodorant.

The invention is a cooling, soothing, energizing rubefacient and non-oily spa treatment for the feet and body.

The invention may be applied to cotton puffs or transferred to a spray bottle and applied to oily hair and scalp, and then dried thoroughly with a cool (not hot) air hair drier, to reduce oiliness, freshen and deodorize hair and scalp, and to relieve dandruff and itchy scalp. Caution should be applied in this use of Peppermint Isopropyl Alcohol, which is flammable. A hot air hair drier must be totally avoided.

The invention may be applied to cotton puffs to be used for cleaning, freshening and deodorizing the insides of shoes, boots, sneakers and slippers. It is harmless to leather, imitation leather and other shoe linings.

The compound is not to be swallowed or used in the eyes or on anal and genital areas. If swallowed, the user should immediately seek assistance from a physician or poison control center. If the compound is used on an area where it causes burning or itching, the area should immediately be flushed with plain water.

The invention is less costly than more complex alcohol toiletries.

The claims are:

1. An improvement on plain isopropyl alcohol containing:
a. 70% to 91% isopropyl alcohol
b. pure, natural peppermint oil
c. natural pink coloring produced by the addition and blending in of FD&C red and FD&C blue liquid colorings,
whereby the resulting solution my be used for stimulating, freshening, cooling, itch-relieving, and deodorizing properties not characteristic of plain alcohol, and my be distinguished, by its color, from plain alcohol and wintergreen alcohol.

2. An improvement on natural peppermint oil containing:
a. an isopropyl alcohol solvent that dilutes and reduces the potency of peppermint oil, whereby the resulting solution is a non-oily, antimicrobial peppermint therapy for feet, body, hair and scalp, and a deodorizing, sanitizing and fragrant cleanser for shoe linings.
